(19) United States
(12) Patent Application Publication
(10) Pub. No.: US 2019/0153377 A1
(43) Pub. Date: May 23, 2019

(54) STERILE CONNECTOR AND CELL CULTURE DEVICE PROVIDED THEREWITH

(71) Applicant: Hitachi High-Technologies Corporation, Tokyo (JP)

(72) Inventors: Akihiro SHIMASE, Tokyo (JP); Eiichiro TAKADA, Tokyo (JP); Kazumichi IMAI, Tokyo (JP); Toshinari SAKURAI, Tokyo (JP)

(21) Appl. No.: 15/521,348

(22) PCT Filed: Nov. 7, 2014
(86) PCT No.: PCT/JP2014/079537

§ 371 Date: Apr. 24, 2017

(51) Int. Cl. C12M 1/12; C12M 1/00; C12M 3/00

(52) U.S. Cl. CPC C12M 37/04; C12M 23/44; C12M 23/34

(57) ABSTRACT

A sterile connector has a first connector including a first housing provided with a first flow channel; a first pipeline connected to the first flow channel; first and second openings, the first opening being positioned inward from the second opening; and a first sealing member covering the second opening. Also, a second connector includes a second housing provided with a second flow channel; a third opening; and a second sealing member covering the third opening. The first and second connectors are detachable from each other. The first sealing member seals a space between the inner circumferential surface of the first housing and the outer circumferential surface of the second housing, while the second sealing member seals a space between the inner circumferential surface of the second housing and the outer circumferential surface of the first pipeline so that the first flow channel communicates with the second flow channel.

### TECHNICAL FIELD

The present invention relates to a sterile connector and a cell culture device provided therewith, particularly to a sterile connector, which is suitable to sterilely detach a cell culture vessel or the like from a closed cell culture device, and a cell culture device provided with the sterile connector.

### BACKGROUND ART

In cell culture, in order to prevent contamination from outside, a closed cell culture device, in which a culture medium bag or a drainage bag is connected to a closed cell culture vessel via a tube and culture is performed in such a closed system, is commonly used.

In addition, a structure disclosed in PTL 1 has been proposed as a sterile connector assembly that is capable of connecting two different flow channels while preventing contamination from outside. The sterile connector assembly is configured to include a first connector and a second connector, the first connector being provided with a stem demarcating a flow channel in the connector, a first housing surrounding the stem and demarcating a first opening, and a first valve disposed on the first opening. In addition, the second connector is provided with a second housing, which is configured to be fitted into the first housing and demarcates a second opening, and a second valve disposed on the second opening. In such a configuration, when the first housing and the second housing engage with each other, the first valve and the second valve engage with each other.

### CITATION LIST

### Patent Literature

PTL 1: JP-T-2011-515197

### SUMMARY OF INVENTION

### Technical Problem

However, in the sterile connector assembly disclosed in PTL 1, the first valve of the first connector and the second valve of the second connector are brought into connect with each other, then, the stem in the first connector is caused to move to the second connector side, thereby, the stem pushes and opens the first valve and the second valve, the first valve and the second valve engage with each other to be folded, and thereby sealing is performed.

Hence, only a push of one of the first connector or the second connector to the other one side does not enable the first valve and the second valve to engage with each other, and thus it is necessary to perform two stages of operations of the push and moving of the stem in the first connector. If a procedure of the two stages of operations is incorrectly understood, that is, if the first connector is pulled from the second connector, there is a concern that it is not possible to seal the flow channels by the first and second valves, respectively, and this results in invasion of particles including microorganisms, types of bacteria, or the like from outside. In addition, since it is necessary to perform the two stages of operations, it is not possible to expect improvement of workability by operators.

On the other hand, in a clinical trial of a cornea transplant using cell sheets, as a procedure, on the previous day, a cell sheet in a cell culture vessel is detached and examined during culture in a plurality of cell culture vessels. Then, in a case where multiple cells are cultured, detaching and examining of some of cultured cells are considered, in order to check a quality of the cultured cells . In the closed cell culture device in which the plurality of cell culture vessels are connected, it is desirable to detach and check cells in a manner described above while the entire closed system is maintained.

According to the invention, there is provided a sterile connector, which is suitable for easily detaching a desired cell culture vessel from a closed cell culture device while particles including microorganisms, types of bacteria, or the like are prevented from invading the device from the outside world, and a cell culture device provided with the sterile connector.

### Solution to Problem

In order to solve such a problem, a sterile connector according to the invention includes: (1) a first connector that is provided with a first housing having a first flow channel for causing a fluid to flow therethrough, a first pipeline which is continuous to the first flow channel, a first opening to which one end of the first pipeline is opened, a second opening which is demarcated with an end portion of the first housing, and a first sealing member which covers the second opening with the first opening positioned inward from the second opening in an axial direction of the first housing; and (2) a second connector that is provided with a second housing having a second flow channel for causing a fluid to flow therethrough, a third opening which is demarcated with an end portion of the second housing, and a second sealing member which covers the third opening. The first and second connectors are attachable to and detachable from each other. The first sealing member seals a gap between an outer circumferential surface of the second housing and an inner circumferential surface of the first housing with which the second opening is demarcated. The second sealing member seals a gap between an outer circumferential surface of the first pipeline and an inner circumferential surface of the second housing with which the third opening is demarcated, and the first flow channel communicates with the second flow channel through the second sealing member.

In addition, a cell culture device according to the invention includes: a cell culture vessel that is provided with an inflow channel through which a liquid for culture circulates and an outflow channel through which the liquid is discharged after use; and an integrated flow-channel member that is configured to connect a plurality of cell culture vessels in parallel, that is provided with an upstream-side divergence flow channel and a downstream-side divergence flow channel which correspond to the cell culture vessels, and that sends a liquid for culture to any desired cell culture vessel of the plurality of cell culture vessels, that is, to the inflow channel via the upstream-side divergence flow channel. The integrated flow-channel member is provided with a first housing having a first pipeline that is continuous to each of the upstream-side divergence flow channel and the downstream-side divergence flow channel, a first opening to which one end of the first pipeline is opened, a second opening which is demarcated with an end portion of the first housing, and a first sealing member which covers the second opening. The cell culture vessel is provided with a second housing having each of the inflow channel and the outflow channel, a third opening which is demarcated with an end portion of the second housing, and a second sealing member which covers the third opening. The first sealing member seals a gap between an inner circumferential surface of the first housing and an outer circumferential surface of the second housing, the second sealing member seals a gap between the second housing and an outer circumferential surface of the first pipeline, and the cell culture vessel is connected to the integrated flow-channel member.

In addition, a cell culture vessel according to the invention includes: a cell culture vessel that is provided with an inflow channel through which a liquid for culture circulates and an outflow channel through which the liquid is discharged after use; and an integrated flow-channel member that is configured to connect a plurality of cell culture vessels in parallel, that is provided with an upstream-side divergence flow channel and a downstream-side divergence flow channel which correspond to the cell culture vessels, and that sends a liquid for culture to any desired cell culture vessel of the plurality of cell culture vessels, that is, to the inflow channel via the upstream-side divergence flow channel. The cell culture vessel is provided with a first housing having a first pipeline that is continuous to each of the inflow channel and the outflow channel, a first opening to which one end of each of the first pipelines is opened, a second opening which is demarcated with an end portion of the first housing, and a first sealing member which covers the second opening. The integrated flow-channel member is provided with a second housing having each of the upstream-side divergence flow channel and the downstream-side divergence flow channel, a third opening which is demarcated with an end portion of the second housing, and a second sealing member which covers the third opening. The first sealing member seals a gap between an inner circumferential surface of the first housing and an outer circumferential surface of the second housing, the second sealing member seals a gap between the second housing and an outer circumferential surface of the first pipeline, and the cell culture vessel is connected to the integrated flow-channel member.

### Advantageous Effects of Invention

According to the invention, it is possible to provide the sterile connector, which is suitable to easily detach a desired cell culture vessel from the closed cell culture device while particles including microorganisms, types of bacteria, or the like are prevented from invading the device from the outside world, and the cell culture device provided with the sterile connector.

Problems, configurations, and effects other than the problems, configurations, and effects described above are clarified in the following description of embodiments.

### BRIEF DESCRIPTION OF DRAWINGS

FIG. 1 illustrates views of a schematic configuration of a sterile connector of Example 1 according to one Example of the invention, FIG. 1(a) illustrates a state in which a first connector and a second connector are connected to each other, FIG. 1(b) illustrates a state of an intermediate stage of pulling the second connector from the first connector, and FIG. 1(c) illustrates a state in which the second connector is completely pulled out from the first connector.

FIG. 2 illustrates longitudinal-sectional views of a sterile connector of Example 2 according to another Example of the invention, FIG. 2(a) illustrates a state in which a first connector and a second connector are connected to each other, FIG. 2(b) illustrates a state of an intermediate stage of pulling the second connector from the first connector, and FIG. 2(c) illustrates a state in which the second connector is completely pulled out from the first connector.

FIG. 3 illustrates views of the external appearance of the sterile connector illustrated in FIG. 2, FIG. 3(a) is a view illustrating the external appearance of a sealing member, FIG. 3(b) is a view illustrating the external appearances of the first and second connectors, and FIG. 3(c) is an exploded perspective view illustrating the first and second connectors.

FIG. 4 illustrates longitudinal-sectional views of a sterile connector of Example 3 according to another Example of the invention, FIG. 4(a) illustrates a state in which a first connector and a second connector are connected to each other, FIG. 4(b) illustrates a state of an intermediate stage of pulling the second connector from the first connector, and FIG. 4(c) illustrates a state in which the second connector is completely pulled out from the first connector.

FIG. 5 is a longitudinal-sectional view of a first connector of Example 4 according to another Example of the invention.

FIG. 6 is a view illustrating an entire schematic configuration of a cell culture device of Example 5 according to another Example of the invention.

FIG. 7 is a perspective view of the external appearances of a cell culture vessel and an integrated flow-channel member which configure the cell culture device illustrated in FIG. 6.

FIG. 8 is a longitudinal-sectional view of the cell culture vessel and the integrated flow-channel member illustrated in FIG. 7.

FIG. 9 is an enlarged view of region B illustrated in FIG. 8, and is a partially enlarged view of a connection portion between the cell culture vessel and the integrated flow-channel member.

FIG. 10 is a longitudinal-sectional view of the integrated flow-channel member illustrated in FIG. 8, and is a view for illustrating an operation of a flow-channel switching member.

FIG. 11 is a partial longitudinal-sectional view illustrating the connection portion between the cell culture vessel and the integrated flow-channel member illustrated in FIG. 8, and a view illustrating a structure of a fixing portion.

FIG. 12 illustrates views of a modification example of the cell culture vessel and the integrated flow-channel member that configure the cell culture device illustrated in FIG. 7, FIG. 12(a) is a top view of the cell culture vessel, and FIG. 12(b) is a perspective view of the external appearances of the cell culture vessel and the integrated flow-channel member.

### DESCRIPTION OF EMBODIMENTS

Hereinafter, Examples of the invention will be described with reference to the accompanying figures.

### Example 1

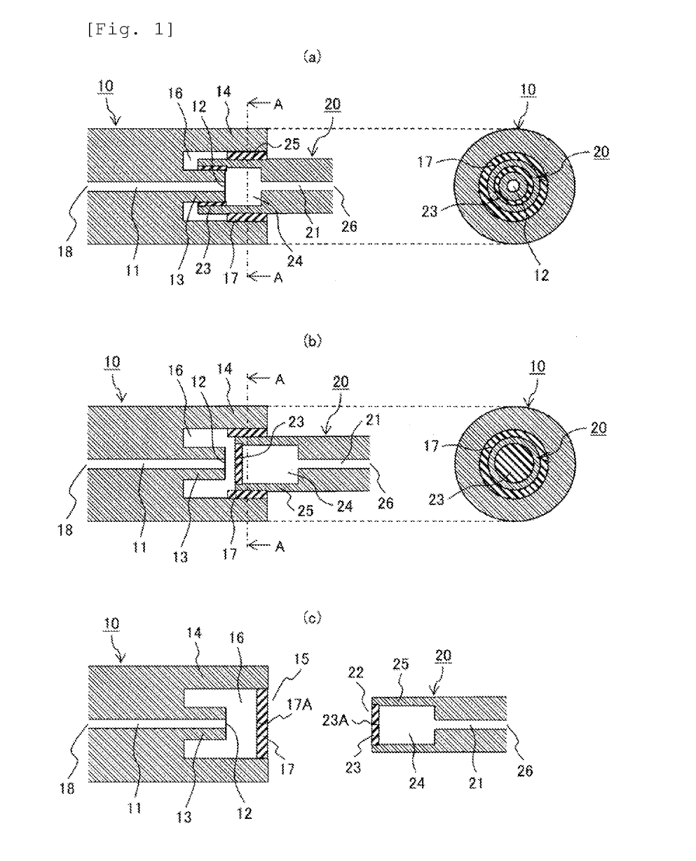

FIG. 1 illustrates views of a schematic configuration of a sterile connector of Example 1 according to one Example of the invention, FIG. 1(a) illustrates a state in which a first connector 10 and a second connector 20, which configure the sterile connector, are connected to each other, FIG. 1(b) illustrates a state of an intermediate stage of pulling (detaching) the second connector 20 from the first connector 10, and FIG. 1(c) illustrates a state in which the second connector 20 is pulled out (detached) from the first connector 10 and the connectors are separated from each other.

As illustrated in FIG. 1(c), the first connector 10 includes, in a cylindrical first housing 14, a first flow channel 11 in which a fluid circulates, a second opening 15 that is demarcated with the first housing 14 at one end of the connector, a first connector end 18 at the other end thereof, and a cylindrical first pipeline 13 that is formed at (formed to be continuous to) one end portion of the first flow channel 11 and has an outer diameter smaller than an inner diameter of the first housing 14.

A front end portion of the first pipeline 13 is positioned on the inner side in an axial direction of the first connector 10 by a predetermined distance from an end portion of the first housing 14 with which the second opening 15 is demarcated, in the axial direction of the first connector 10. In addition, the front end portion of the first pipeline 13 is opened to form a first opening 12, and the first opening 12 communicates with the first flow channel 11. An outer circumferential surface of the first pipeline 13 and an inner circumferential surface of the first housing 14 form a first recessed portion 16 as a space having a recess-shaped longitudinal section. In other words, in a columnar space that is continuous from the end portion of the first housing 14, with which the second opening 15 is demarcated, to the inside of the first connector 10 in the axial direction thereof, the first pipeline 13 having the outer diameter smaller than the inner diameter of the first housing 14 has the first flow channel 11 inside and is formed to have a projecting-shaped longitudinal section so as to project toward the second opening 15 side.

A disk-shaped first sealing member 17 is provided to close the second opening 15, at the end portion of the first housing 14 with which the second opening 15 is demarcated. The disk-shaped first sealing member 17 has an outer circumferential surface that is fixed to the inner circumferential surface of the first housing 14, and is provided with a line of a first slit 17A at the substantially central portion.

In addition, the second connector 20 includes, in a cylindrical second housing 25, a second flow channel 21 in which a fluid circulates, a third opening 22 that is demarcated with the second housing 25 at one end of the connector, and a second connector end 26 at the other end thereof. A second recessed portion 24 as a cylindrical space, which is demarcated with the inner circumferential surface of the second housing 25, is formed to be continuous from an end portion of the second housing 25, with which the third opening 22 is demarcated, to a front end portion of the second flow channel 21 in the axial direction of the second connector 20. An inner diameter of the second housing 25 with which the second recessed portion 24 is demarcated is larger than a diameter of the second flow channel 21, and the diameter of the first flow channel 11 is substantially equal to the diameter of the second flow channel 21. A disk-shaped second sealing member 23 is provided to close the third opening 22, at the end portion of the second housing 25 with which the third opening 22 is demarcated. The disk-shaped second sealing member 23 has an outer circumferential surface that is fixed to the inner circumferential surface of the second housing 25, and is provided with a line of a second slit 23A at the substantially central portion. Here, the outer circumferential surface of the first sealing member 17 and the inner circumferential surface of the first housing 14 are fixed, and the outer circumferential surface of the second sealing member 23 and the inner circumferential surface of the second housing 25 are fixed through adhesion with an adhesive, heat welding, or the like. When the adhesion is performed, it is desirable to employ an adhesion method which does not influence cells by using an adhesive without cytotoxicity or the like.

An outer diameter of the first pipeline 13 of the first connector 10 is smaller than an inner diameter of the end portion of the second housing 25 with which the third opening 22 of the second connector 20 is demarcated. In addition, an outer diameter of the second housing 25 of the second connector 20 is smaller than the inner diameter of the first housing 14 with which the second opening 15 of the first connector 10 is demarcated.

In addition, it is preferable that the first sealing member 17 and the second sealing member 23 are made of a material that has elasticity and high adhesiveness and is sterilizable, and, for example, it is suitable to use an elastic material such as rubber. In addition, it is desirable that members that form the first flow channel 11 and the second flow channel 21, that is, the first housing 14 and the second housing 25, are made of plastic such as polycarbonate, polystyrene, or polypropylene which has plasticity and stiffness without cytotoxicity. Instead of the plastic, the members may be made of metal without cytotoxicity.

Next, a pulling (detaching) operation of the sterile connector configured to include the first connector 10 and the second connector 20 will be described.

The left figure in FIG. 1(a) illustrates a longitudinal-sectional view obtained in a state in which the first connector 10 and the second connector 20 are connected to or are engaged with each other, and the right figure illustrates a cross-sectional view along A-A in an arrow direction. One of the first connector 10 and the second connector 20 is pushed to the other one side, and thereby the connectors enter a connection or engagement state. Hereinafter, as an example, a case where the first connector 10 is fixed and the second connector 20 is caused to move will be described.

As illustrated in the left figure in FIG. 1(a), a gap between the outer circumferential surface of the first pipeline 13 that configures the first connector 10 and the inner circumferential surface of the second housing 25, with which the second opening 22 of the second connector 20 is demarcated, is sealed with the second sealing member 23. In addition, a gap between the outer circumferential surface of the second housing 25 of the second connector 20 and the inner circumferential surface of the first housing 14, with which the second opening 15 of the first connector 10 is demarcated, is sealed with the first sealing member 17. The first flow channel 11 communicates with the second flow channel 21 via the second recessed portion 24. At this time, as illustrated in the right figure in FIG. 1(a), the first pipeline 13, the second sealing member 23, the second housing 25, the first sealing member 17, and the first housing 14 are arranged concentrically in a radial direction with the first opening 12 as the center, and the first sealing member 17 and the second sealing member 23 ensure sealability. In other words, it is possible for the first flow channel 11 to communicate with the second flow channel 21 while maintaining a closed system. A front end portion of the second housing 25 is inserted to the first connector 10 side via a line of the first slit 17A provided at the substantially central portion of the first sealing member 17 as described above, and thereby the first sealing member 17 is pushed into the inner circumferential surface side of the first housing 14. The second sealing member 23 is pushed into the inner circumferential surface side of the second housing 25 by the front end portion of the first pipeline 13 via a line of the second slit 23A provided at the substantially central portion of the second sealing member 23.

When the second connector 20 is caused to move in a rightward direction in a view toward the paper surface from the connection or engagement state illustrated in FIG. 1(a), the connector enters an intermediate state in which the second connector 20 is pulled (detached) from the first connector 10 as illustrated in FIG. 1(b). As illustrated in the left figure in FIG. 1(b), the end portion of the second housing 25, with which the third opening 22 is demarcated, moves and is separated in the rightward direction in a view toward the paper surface from the front end portion of the first pipeline 13, that is, the first opening 12. At this time, the first pipeline 13, by which the second sealing member 23 is pushed into the inner circumferential surface side of the second housing, is separated from the end portion of the second housing 25, and thereby, for example, the second sealing member 23 formed of the elastic material such as rubber enters a state of sealing the third opening 22 due to own elastic force. In this state, the front end portion of the second housing 25, with which the third opening 22 is demarcated, is still positioned inside the first housing 14, with which the second opening 15 is demarcated. In this manner, a gap between the outer circumferential surface of the second housing 25 and the inner circumferential surface of the first housing 14 is maintained to be in a state of being sealed with the first sealing member 17. As illustrated in the right figure in FIG. 1(b), the second housing 25, the first sealing member 17, and the first housing 14 are arranged concentrically in a radial direction with the second sealing member 23 as the center, the third opening 22 is sealed with the second sealing member 23, and thereby the second flow channel 21 is in an airtight (closed) state. The right figure in FIG. 1(b) does not illustrate the second slit 23A; however, the third opening 22 automatically enters an airtight state due to the elastic force of the second sealing member 23 as described above. At this time, the second slit 23A, via which the first pipeline 13 is inserted thereto, is closed due to the elastic force and thus is not illustrated in the figure.

When the second connector 20 is caused to further move in the rightward direction in a view toward the paper surface from the state illustrated in FIG. 1(b), the connector has a state illustrated in FIG. 1(c). The left figure in FIG. 1(c) illustrates a state obtained after the second connector 20 is pulled out from the first connector 10. When the second connector 20 is caused to further move in the rightward direction in a view toward the paper surface from the state illustrated in FIG. 1(b) , the end portion of the second housing 25, with which the third opening 22 is demarcated, is separated from the end portion of the first housing 14, with which the second opening 15 is demarcated. At this time, the end portion of the first housing 14, with which the second opening 15 is demarcated, is separated from the second housing 25 by which the first sealing member 17 is pushed into the inner circumferential surface side of the first housing 14 thereto. In this manner, the first sealing member 17 made of the elastic material such as rubber enters a state of sealing the second opening 15 due to own elastic force. Similarly to the second sealing member 23, the first slit 17A is closed due to the elastic force. In this manner, it is possible for the first connector 10 and the second connector 20 to be detached while the closed system is maintained with the first sealing member 17 and the second sealing member 23, respectively.

In this Example, the example in which the first connector 10 is fixed and the second connector 20 is caused to move is described; however, instead of this, even when the second connector 20 is fixed and the first connector 10 is caused to move, the same function as described above is achieved.

According to this Example, it is possible to detach the first connector 10 provided with the first flow channel 11 and the second connector 20 provided with the second flow channel 21 while the closed system is maintained.

In addition, while particles containing microorganisms or bacteria are prevented from invading the first flow channel 11 or the second flow channel 21 from the outside world, it is possible to realize the sterile connector in which it is possible to perform detachment by just moving of one connector in one direction.

### Example 2

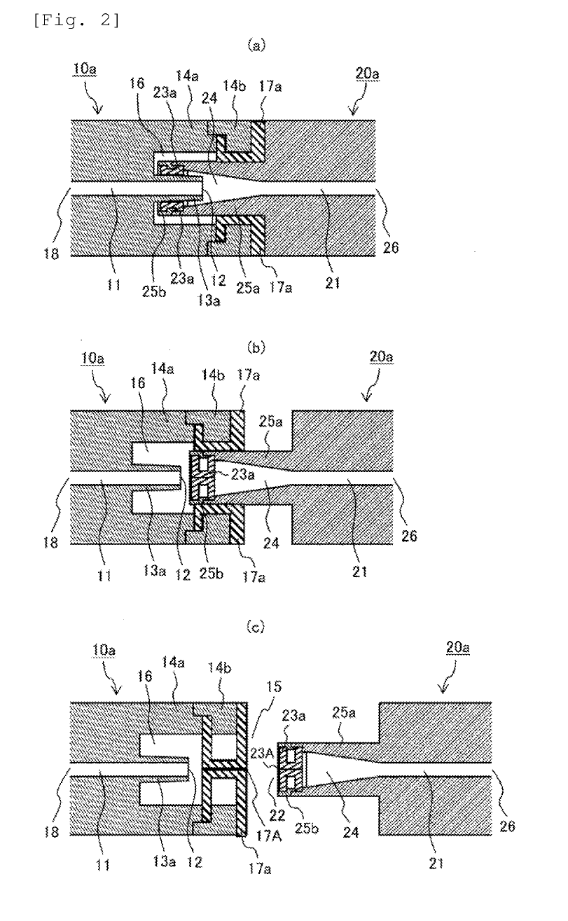

FIG. 2 illustrates longitudinal-sectional views of a sterile connector of Example 2 according to another Example of the invention. FIG. 2(a) illustrates a state in which a first connector and a second connector are connected to each other, FIG. 2(b) illustrates a state of an intermediate stage of pulling the second connector from the first connector, and FIG. 2(c) illustrates a state in which the second connector is completely pulled out from the first connector. In Example 1,the first sealing member 17 and the second sealing member 23 are directly fixed to the inner circumferential surfaces of the first housing 14 and the second housing 25, respectively, through adhesion with an adhesive or heat welding. In this Example, the first and second housings are provided with a structure of holding the sealing members and a shape or the like of the sealing member is different from that of Example 1.The same reference signs are assigned to the same components as those in FIG. 1, and the description thereof will be omitted below.

As illustrated in FIG. 2(c), a first sealing member 17a has a substantially H-shaped longitudinal section, and an outer edge portion of a disk-shaped portion thereof on the first pipeline 13a side is interposed and fixed between two divided first housings 14a and 14b. The two divided first housings 14a and 14b are subjected to adhesion or heat welding to each other on the outer circumference sides, respectively. In addition, the first pipeline 13a has a shape formed with the outer diameter thereof increasing to the left side from the first opening 12 in an axial direction of a first connector 10a. In other words, the outer circumferential surface of the first pipeline 13 is inclined similar to the side surface of a cone. In this manner, resistance is reduced when the first pipeline 13 is inserted via a line of the second slit 23A provided in a second sealing member 23a which will be described below.

In addition, a second sealing member 23a has a substantially H-shaped longitudinal section, and an outer edge portion of a disk-shaped portion thereof on the second flow channel 21 side is interposed and fixed between two divided second housings 25a and 25b. The two divided second housings 25a and 25b are subjected to the adhesion or the heat welding to each other on the outer circumference sides, respectively. The second recessed portion 24 demarcated with the inner circumferential surface of the divided second housing 25a has a shape formed with a diameter of the inner circumferential surface of the divided second housing 25a reduced from the second sealing member 23a side to the front end portion of the second flow channel 21. In this manner, in a state in which the first connector 10a is connected to or engaged with the second connector 20a as illustrated in FIG. 2(a), the diameter of the second recessed portion 24 that communicates with the first flow channel 11 and the second flow channel 21, that is, an inner diameter of the second housing 25a can approximate to a flow-channel diameter of the first flow channel 11 and the second flow channel 21. With a fluid that flows from the first flow channel 11 to the second flow channel 21, or a fluid that flows from the second flow channel 21 to the first flow channel, it is possible to reduce a region in which a flow channel rapidly expands, and it is possible to have uniform flow-channel resistance in the first flow channel 11 and the second flow channel 21 which communicate with each other, compared to Example 1.

In addition, as illustrated in FIG. 2(c), the two-divided second housing 25a has a region containing the second recessed portion 24 and a region containing the second flow channel 21. An outer diameter of the region containing the second recessed portion 24 is smaller than an outer diameter of the region containing the second flow channel 21, and the second housing 25a has a step portion at a position corresponding to a connection portion between the second recessed portion 24 and the second flow channel 21.

In the joining or engagement state illustrated in FIG. 2(a), a gap between the outer circumferential surface of the first pipeline 13a and the inner circumferential surfaces of the second housings 25a and 25b is sealed with the second sealing member 23a. In addition, a gap between outer circumferential surfaces of the two divided second housings 25a and 25b and the inner circumferential surface of the first housing 14 is sealed with the first sealing member 17a. At this time, the disk-shaped portion of the first sealing member 17a on the second opening 15 side is subjected to elastic deformation when the second housing 25a containing the second recessed portion 22 is inserted into the first slit 17A, and the thickness of the portion increases. In addition, the disk-shaped portion of the first sealing member 17a on the second opening 15 side is brought into close contact with the step portion of the second housing 25a, and thereby it is possible to improve airtightness between the first connector 10a and the second connector 20a, compared to Example 1.

In the state illustrated in FIG. 2(b), similar to Example 1, the third opening 22 of the second connector 20a automatically enters an airtight state due to own elastic force of the second sealing member 23. Similarly, in the state illustrated in FIG. 2(c), the second opening 15 of the first connector 10a automatically enters an airtight state due to own elastic force of the first sealing member 17.

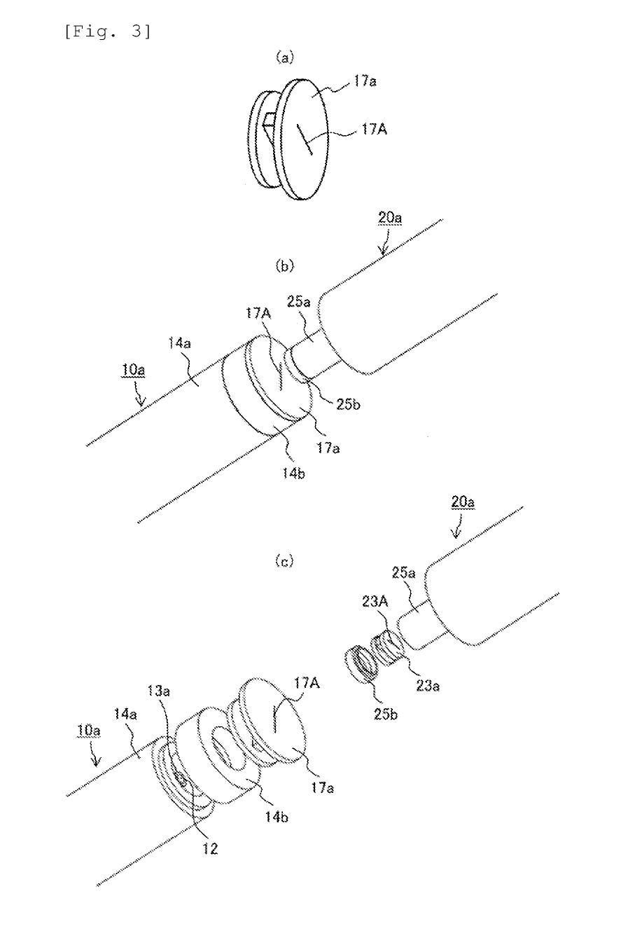

FIG. 3 illustrates views of the external appearance of the sterile connector illustrated in FIG. 2, FIG. 3(a) is a view illustrating the external appearance of the first sealing member 17, FIG. 3(b) is a view illustrating the external appearances of the first and second connectors, and FIG. 3(c) is an exploded perspective view illustrating the first and second connectors. As illustrated in FIG. 3(a), the first sealing member 17a having the H-shaped longitudinal section described above is provided with two disk-shaped portions having different diameters and a connection portion that connects the disk-shaped portions, and the first slit 17A is formed to penetrate through the two disk-shaped portions and the connection portion that connects the disk-shaped portions. In addition, as illustrated in FIGS. 3(b) and 3(c), the first sealing member 17a is assembled to be interposed between the two divided first housings 14a and 14b, as described above. In addition, the second sealing member 23a is assembled to be interposed between the two divided second housings 25a and 25b. In FIG. 2, the description above indicates that the two divided first housings 14a and 14b are subjected to adhesion or heat welding to each other on the outer circumference sides, respectively; however, the following configuration may be employed instead of such a configuration described above. For example, as illustrated in FIG. 3(c), a male screw and a female screw are respectively formed on the inner circumferential surface of the two-divided first housing 14b and the outer circumferential surface of the first housing 14a having a projecting shape and a cylindrical shape and including a surface that supports an outer edge portion of the disk-shaped portion having a small diameter which configures the first sealing member 17a, and the first housings 14a and 14b are screwed. Similarly, a configuration in which the two divided second housings 25a and 25b are screwed to each other may be employed. In order to further improve the airtightness of the screwed portions, the screwing may be performed after an adhesive agent without the cytotoxicity is applied on the screw portion.

According to this Example, the inclination on the outer circumferential surface of the first pipeline 13 enables the resistance to be reduced during the insertion of the second sealing member 23a into the second slit 23A, in addition to the effect of Example 1.

In addition, it is possible to have the uniform flow-channel resistance in the first flow channel 11 and the second flow channel 21 which communicate with each other, compared to Example 1.

### Example 3

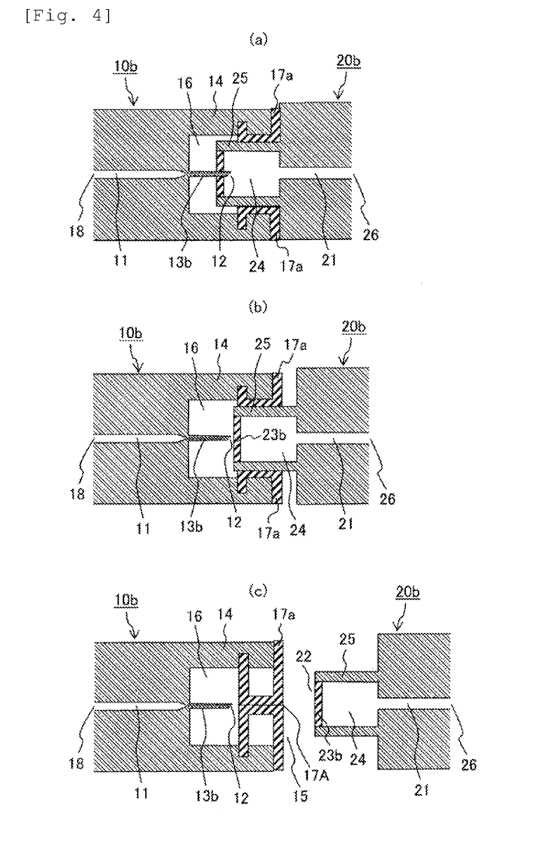

FIG. 4 illustrates longitudinal-sectional views of a sterile connector of Example 3 according to another Example of the invention, FIG. 4(a) illustrates a state in which the first connector and the second connector are connected to each other, FIG. 4(b) illustrates a state of an intermediate stage of pulling the second connector from the first connector, and FIG. 4(c) illustrates a state in which the second connector is completely pulled out from the first connector. This Example differs from Example 1 and Example 2 described above in that the first pipeline 13a that configures the first connector 10a of Example 2 has a needle shape, the shape of the second connector is formed to have an outer diameter of the second housing different from the shape of the second connector 20 illustrated in Example 1 in the region containing the second recessed portion 24 and the region containing the second flow channel. The same reference signs are assigned to the same components as those in Example 1 or Example 2, and the description thereof will be omitted below.

In the states illustrated in FIGS. 4(a) and 4(b), similar to FIGS. 2(a) and 2(b), in the related art, the disk-shaped portion of the first sealing member 17a on the second opening 15 side has a thickness that increases due to the elastic deformation; however, the description thereof is omitted in FIG. 4.

In the joining or engagement state illustrated in FIG. 4(a), a second sealing member 23b of this Example is not provided with a slit, unlike Example 1 and Example 2. The needle-shaped first pipeline 13b penetrates through the second sealing member 23b, and thereby the first flow channel 11 communicates with the second flow channel 22 via the second recessed portion 22. The sealing (airtight closing) between the first housing 14 and the second housing 25 is the same as that in Example 2.

In the state illustrated in FIG. 4(b), when the needle-shaped first pipeline 13b is pulled from the second sealing member 23b, a fine hole formed in the second sealing member 23b, which corresponds to the outer diameter of the first pipeline 13b, is closed due to the own elastic force of the second sealing member 23b. A state in FIG. 4(c) is the same as the state in Example 2 described above, and thus the description thereof is omitted.

In the state illustrated in FIG. 4(b), if pressure is applied to the fluid in the first flow channel 11, for example, the liquid, the liquid leaks to a space of the first recessed portion 16. Also in such a case, the first sealing member 17a and the second sealing member 23b allow airtightness of the inside of the first recessed portion 16, and sterility is maintained because the closed system is maintained. However, it is possible for a liquid to be attached to outer surfaces of the second housing 25 and the second sealing member 23b which configure the second connector 20b, which are positioned in the space of the first recessed portion 16 from the first sealing member 17a. Hence, when the second connector 20b is detached from the first connector 10b, in particular, in a case where there is no need to add pressure to the liquid in the first flow channel 11, it is desirable to perform an operation of detaching the connector without applying pressure. As described above, in the case where there is no need to apply pressure to the liquid in the first flow channel 11, the configuration in which the operation of detaching the connector is performed without applying pressure thereto is not limited to the configuration of the first connector 10b and the second connector 20b illustrated in FIG. 4, the invention may also be applied to the structure of the sterile connector of Example 1 described above, that is, the configuration of the first connector 10 and the second connector 20 illustrated in FIG. 1. Similarly, the invention may be applied to the structure of the sterile connector of Example 2 described above, that is, the structure of the first connector 10a and the second connector 20a illustrated in FIG. 2.

When pressure of the liquid in the first flow channel 11 is the negative pressure, that is, the first flow channel equivalently enters a suction state, and thereby it is possible to prevent the liquid from leaking to the space of the first recessed portion 16 from the needle-shaped first pipeline 13b. In this case, elastic tubes (not illustrated) are connected to the first connector end 18 of the first connector 10b and the second connector end 26 of the second connector 20b, respectively, and a pinch valve is connected to one elastic tube and a squeeze pump is connected to the other elastic tube. In the connection state illustrated in FIG. 4(a), the pinch valve is closed, and the squeeze pump is driven such that the liquid flows from the second flow channel 21 to the first flow channel 11. The squeeze pump is driven for a certain time and the pressure in the first flow channel 11 becomes a desired negative pressure. Then, as illustrated in FIGS. 4(b) and 4(c), when the second connector 20b is detached from the first connector 10b, it is possible to prevent the liquid from leaking from the first opening 12 of the first flow channel 11. The configuration, in which the pressure of the liquid in the first flow channel is the negative pressure, is not limited to the configuration of the first connector 10b and the second connector 20b illustrated in FIG. 4, but the invention may also be applied to the configurations of the first connector 10 and the second connector 20 illustrated in FIG. 1 described above, and the first connector 10a and the second connector 20a illustrated in FIG. 2.

According to this Example, since there is no need to form a slit in the second sealing member 23b, it is possible to further improve the airtightness (closedness) of the second connector 20b, in addition to effects of Example 1.

### Example 4

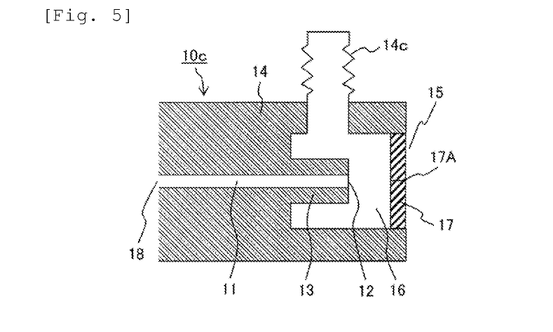

FIG. 5 is a longitudinal-sectional view of a first connector of Example 4 according to another Example of the invention. This Example differs from Example 1 in that there is provided a mechanism that changes the pressure in a space of the first recessed portion 16 in the first connector 10 described in Example 1 into the positive pressure. The same reference signs are assigned to the same components as those in Example 1, and the description thereof will be omitted below.

As illustrated in FIG. 5, a first connector 10c, which has one end which communicates with the first recessed portion 16 and the other end which is closed, includes, in the first housing 10c, a variable protrusion 14c that forms a closed space integrally with the first recessed portion 16 and has a variable volume of the closed space. The variable protrusion 14c has a bellows structure. The variable protrusion 14c is pressed by an operator, thereby the volume of the closed space formed by the variable protrusion integrally with the first recessed portion 16 decreases, and the space of the first recessed portion 16 has the positive pressure. In addition, the variable protrusion is drawn by an operator, thereby the volume of the closed space formed by the variable protrusion integrally with the first recessed portion 16 increases, and the space of the first recessed portion 16 has the negative pressure.

When the variable protrusion 14c is pressed down in a state in which the first connector 10c is connected to the second connector 20 illustrated in FIG. 1, the space of the first recessed portion 16 has the positive pressure. When the first connector 10c is pulled from the second connector 20 while this state is maintained, the liquid does not leak from the first flow channel 11 to the space of the first recessed portion 16 from the first pipeline 13 that configures the first connector 10c. Accordingly, it is possible to prevent the liquid from being attached to the outer surfaces of the second sealing member 23 and the end portion (end portion with which the third opening 22 is demarcated) of the second housing 25 which configure the second connector 20.

In this Example, the second connector is the second connector 20 of Example 1; however, the structure is not limited thereto, and the structure may be the second connector 20a illustrated in FIG. 2 or the second connector 20b illustrated in FIG. 4. In addition, the first sealing member 17 and the first pipeline 13 that configure the first connector 10c may be replaced with the first pipeline 13a and the first sealing member 17a illustrated in FIG. 2 or the first pipeline 13b and the first sealing member 17a illustrated in FIG. 4. Even in any case, the variable protrusion 14c may have a structure in which it is possible to form the closed space integrally with the first recessed portion 16.

In addition, instead of the variable protrusion 14c, a configuration in which a port that can communicate with the first recessed portion 16 is provided in the first housing 14, the pump is connected to the port via the elastic tube or the like, and the space of the first recessed portion 16 has the positive pressure state may be employed.

According to this Example, in the case where the first connector is detached from the second connector, it is possible to prevent the liquid in the first flow channel from leaking from the first pipeline that configures the first connector, in addition to the effects of Example 1.

### Example 5

FIG. 6 is a view illustrating an entire schematic configuration of a cell culture device of Example 5 according to another Example of the invention. As illustrated in FIG. 6, a cell culture device 1 includes a cell culture vessel 31, a supply bag 32 that contains a culture medium such as a cell culture solution, a collecting bag 33 that collects the culture medium such as the cell culture solution or the like after use, and a flow-channel switching member 38. The cell culture vessel 31 is connected to the supply bag 32 and the collecting bag 33 via a flow channel. FIG. 6 illustrates an example in which four cell culture vessels 31 are provided; however, the number of cell culture vessels is not limited thereto, and a desired number of cell culture vessels 31 may be provided. In addition, the cell culture device 1 includes an upstream-side common flow channel 34 having one end that is connected to the supply bag 32 and the other end that is connected to upstream-side divergence flow channels 35 (divergence flow channels on the upstream side which are connected to the cell culture vessels 31), a downstream common flow channel 37 having one end that is connected to the collecting bag 33 and the other end that is connected to the flow-channel switching member 38, and downstream-side divergence flow channels 36 (divergence flow channels on the downstream side which are connected to the cell culture vessels 31) which are connected to the flow-channel switching member 38 and the cell culture vessels 31.

Since the cell culture device 1 is a closed culture system, it is necessary to apply the drive force of a liquid such as the cell culture solution as the culture medium from the outside of the closed culture system. The elastic tube and the squeeze pump 39 that sends the liquid in a squeezing manner from the outside are provided in the upstream-side common flow channel 34. Therefore, at least a part of the upstream-side common flow channel 34 needs to be formed of a flow channel having elasticity. The drive of the squeeze pump 39 causes the liquid such as a solution in cells to be pressurized in the upstream-side common flow channel 34, and the liquid flows to the cell culture vessel 31 in the upstream-side common flow-channel 34. In other words, the inside of the flow channel has the positive pressure. Instead of the disposition of the squeeze pump 39 in the upstream-side common flow channel 34, the squeeze pump 39 may be disposed in the downstream-side common flow channel 37. In this case, the inside of the downstream-side common flow channel 37 has the negative pressure, and the liquid such as the cell culture solution as the culture medium is sucked out from the supply bag 32. In addition, the configuration is not limited thereto, and a configuration in which the squeeze pumps 39 are provided to both of the upstream-side common flow channel 34 and the downstream-side common flow channel 37, respectively, may be employed. In this case, the two squeeze pumps 39 provided to the upstream-side common flow channel 34 and the downstream-side common flow channel 37 can reduce a pressure load with respect to the liquid such as the cell culture liquid or the like which flows in the flow channels, respectively.

The drive of the squeeze pump 39 causes the liquid such as the cell culture solution in the supply bag 32 to be sent to the cell culture vessel 31 via the upstream-side common flow channel 34 and the upstream-side divergence flow channel 35. At this time, a switching operation of the flow-channel switching member 38 causes the liquid to be sent to the cell culture vessel 31 that is connected to the downstream-side common flow channel 37. In the cell culture vessel 31, the cell culture solution or the like that remains in the cell culture vessel 31 after use is pushed by the liquid such as the inflow cell culture solution, and is sent to the collecting bag 33 via the downstream-side divergence flow channel 36, the flow-channel switching member 38, and the downstream-side common flow channel 37.

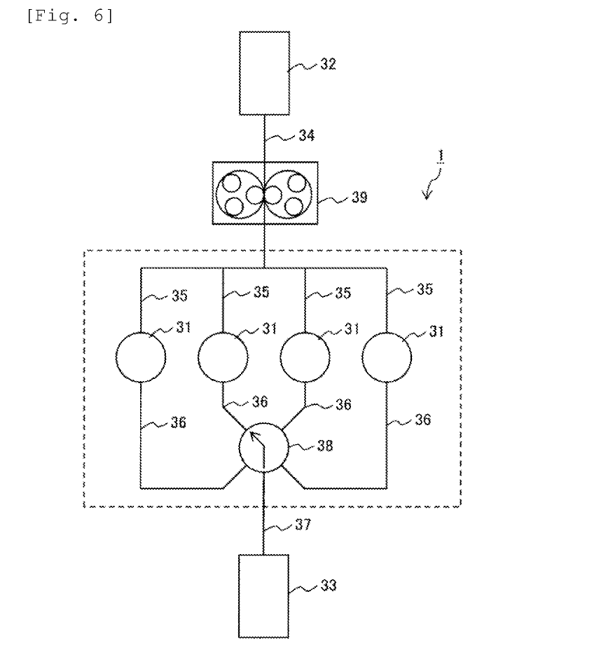

FIG. 7 is a perspective view of the external appearances of an integrated flow-channel member and the cell culture vessel, which configure the cell culture device illustrated in FIG. 6, in which the integrated flow-channel member is formed as an integral member of all of the upstream-side and downstream-side common flow channels, the upstream-side and downstream-side divergence flow channels, and the flow-channel switching member. As illustrated in FIG. 7, the cell culture vessel 41 includes a culture surface 41a, an inflow channel 41b and an outflow channel 41c which will be described below. An integrated flow-channel member 42 includes a flow-channel switching member 43, and is configured to be connected to the four cell culture vessels 41.

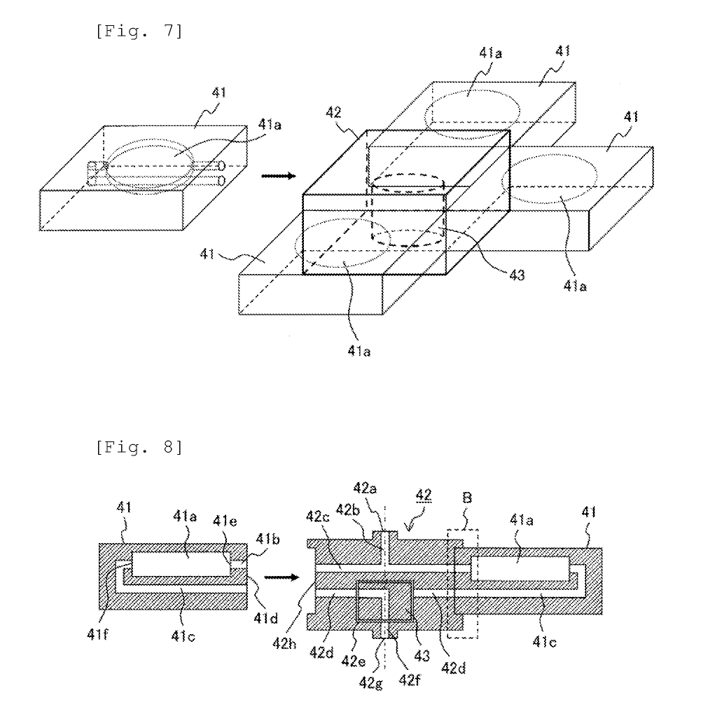

FIG. 8 is a longitudinal-sectional view of the cell culture vessel and the integrated flow-channel member illustrated in FIG. 7. As illustrated in FIG. 8, the cell culture vessel 41 includes the culture surface 41a, the inflow channel 41b, and the outflow channel 41c. The inflow channel 41b communicates with the culture surface 41a at an entrance 41e, and the outflow channel 41c communicates with the culture surface 41a at an exit 41f.

In addition, the integrated flow-channel member 42 includes the inlet 42a in the top surface, the upstream-side common flow channel 42b, the upstream-side divergence flow channel 42c, the downstream-side divergence flow channel 42d, the storage chamber 42e of the flow-channel switching member 43, the downstream-side common flow channel 42f, and the outlet 42g. In addition, the cell culture vessel 41 and the integrated flow-channel member 42 have connection ports 41d and ports 42h, respectively, so as to be connected to each other. The integrated flow-channel member 42 includes a plurality of ports 42h so as to be connected to the plurality of cell culture vessels 41.

For example, it is desirable that the cell culture vessel 41 and the integrated flow-channel member 42 are made of plastic such as polycarbonate, polystyrene, or polypropylene which has plasticity and stiffness without cytotoxicity.

The inflow channel 41b and the outflow channel 41d are connected at the connection port 41d of the cell culture vessel 41, and the inflow channel 41b and the outflow channel 41d are connected to the connection port 41d on one side surface of the cell culture vessel 41. The upstream-side divergence flow channel 42b and the downstream-side divergence flow channel 42d are connected at the port 42h of the integrated flow-channel member 42, and the upstream-side divergence flow channel 42c and the downstream-side divergence flow channel 42d are vertically connected at the ports 42h on the side surfaces of the integrated flow-channel member 42. When the cell culture vessel 41 is connected to the integrated flow-channel member 42, in such a configuration, the upstream-side divergence flow channel 42c communicates with the inflow channel 41b of the cell culture vessel 41, and the downstream-side divergence flow channel 42d communicates with the outflow channel 41c of the cell culture vessel 41.

As described above, in a structure in which the inflow channel 41b and the outflow channel 41d are connected at the connection port 41d on the same side surface of the cell culture vessel 41, and the upstream-side divergence flow channel 42b and the downstream-side divergence flow channel 42d are connected to the ports 42 on the side surfaces of the integrated flow-channel member 42, the cell culture vessel 41 is detached from and is attached to the integrated flow-channel member 42 in one direction, and thus it is easy to perform a detachment/attachment operation.

FIG. 9 is an enlarged view of region B illustrated in FIG. 8, and is a partially enlarged view of a connection portion between the cell culture vessel 41 and the integrated flow-channel member 42. As illustrated in FIG. 8, the second connectors 20 illustrated in FIG. 1 are vertically provided to be continuous to the inflow channel 41b and the outflow channel 41c, respectively, on the same side surface on which the inflow channel 41b and the outflow channel 41c of the cell culture vessel 41 are disposed. In addition, the first connectors 10 illustrated in FIG. 1 are vertically provided to be continuous to the upstream-side divergence flow channel 42c and the downstream-side divergence flow channel 42d, respectively, on the same side surface on which the upstream-side divergence flow channel 42c and the downstream-side divergence flow channel 42d of the integrated flow-channel member 42 are disposed. In this manner, the inflow channel 41b of the cell culture device 41 communicates with the upstream-side divergence flow channel 42c of the integrated flow-channel member 42 via the space of the second recessed portion 24 of the second connector 20 while the closed system is maintained. In addition, the outflow channel 41c of the cell culture device 41 communicates with the downstream-side divergence flow channel 42d of the integrated flow-channel member 42 via the space of the second recessed portion 24 of the second connector 20 while the closed system is maintained.

As described above, in the state in which the cell culture vessel 41 is connected to the integrated flow-channel member 42, a detachment operation of the cell culture vessel 41 will be described below.

When the cell culture vessel 41 is caused to move from the state illustrated in FIG. 9 in the rightward direction in a view toward the paper surface, first, the third opening 22 of the second connector 20 is closed with the second sealing member 23 due to own elastic force as described in FIG. 1(b). In this manner, the inflow channel 41b and the outflow channel 41c of the cell culture vessel 41 are closed in the airtight manner. Subsequently, as described in FIG. 1(c), the second opening 15 of the first connector 10 is closed in the airtight manner due to the own elastic force of the first sealing member 17. In this manner, the upstream-side divergence flow channel 42c and the downstream-side divergence flow channel 42d of the integrated flow-channel member 42 are closed in the airtight manner. According to the cell culture device 1 of this Example, only the pulling of the cell culture vessel 41 from the integrated flow-channel member 42 in one direction enables a desired cell culture vessel 41 to be easily detached while the closed system is maintained.

FIG. 9 illustrates a configuration in which the first connector 10 illustrated in FIG. 1 is provided in the integrated flow-channel member 42, and the second connector 20 is provided in the cell culture vessel 41; however, the configuration is not limited thereto, and the first connector 10 may be provided in the cell culture vessel 41, and the second connector 10 may be provided in the integrated flow-channel member 42. In addition, the first connector 10a and the second connector 20a of Example 2 may be used, or further the sterile connector described in Example 3 or 4 may be used.

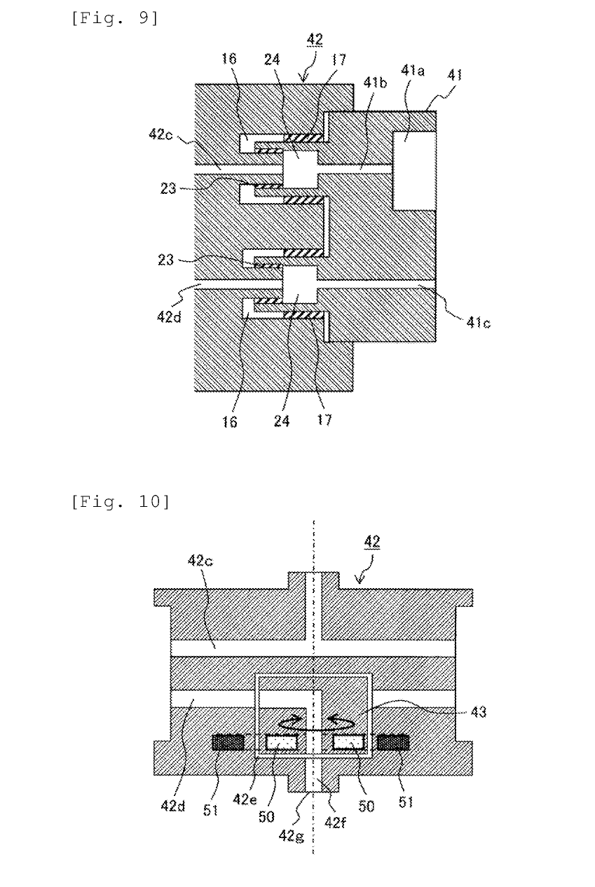

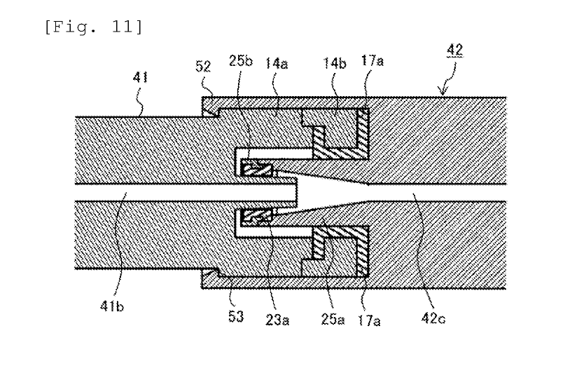

Here, an operation of the flow-channel switching member 43 that configures the integrated flow-channel member 42 is described. FIG. 10 illustrates a longitudinal section of the integrated flow-channel member 42 illustrated in FIG. 8, and is a view for illustrating the operation of the flow-channel switching member 43. As illustrated in FIG. 10, the flow-channel switching member 43, which is disposed in the storage chamber 42e, is provided with a connection flow channel inside so as to communicate with the downstream-side common flow channel 42f and the downstream-side divergence flow channel 42d that communicates with the outflow channel 41c of the desired culture vessel 41. A plurality of permanent magnets 50, which do not interfere with the connection flow channel and are separated from each other to form an annular shape, are embedded. In addition, a plurality of electromagnets 51 formed of a magnetic material around which a coil is wound are embedded in the integrated flow-channel member 42 so as to be separated from each other on the outer side of the storage chamber 42e at positions facing the permanent magnets 50. The coil is energized with a conducting wire (not illustrated), and thereby the flow-channel switching member 43 is caused to rotate. In this manner, the connection flow channel is positioned to face the desired downstream-side divergence flow channel 42d. The conducting wire for energizing the coil is laid around to the outside of the closed cell culture device 1. In addition, a rotating axis of the flow-channel switching member 43 is substantially coincident with the central axis of the downstream-side common flow channel 42f of the integrated flow-channel member 42.

A drive mechanism of the flow-channel switching member 43 is not limited to such a configuration of including the permanent magnets 50 and the electromagnets 51. For example, the flow-channel switching member 43 may be configured to extend to project downward from the integrated flow-channel member 42 (downstream-side common flow channel 42f side) and to transmit a rotational drive force by a stepping motor or a servomotor. In this case, it is necessary to cover the periphery of the projection portion with a film-like sealing member so as to maintain the closed system.

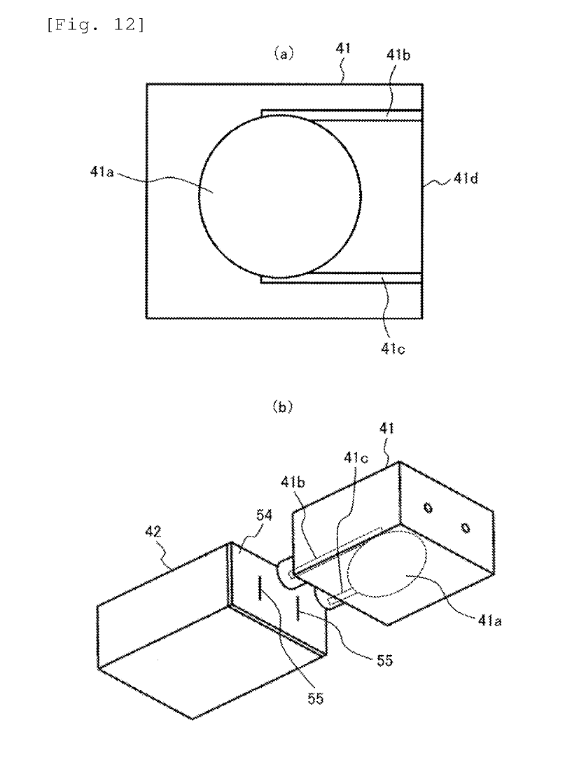

FIG. 11 is a partial longitudinal-sectional view illustrating the connection portion between the cell culture vessel 41 and the integrated flow-channel member 42 illustrated in FIG. 8, and a view illustrating a structure of a fixing portion. FIG. 11 illustrates, as an example, a configuration in which the first connector 10a of Example 2 illustrated in FIG. 2 is provided such that the inflow channel 41b of the cell culture vessel 41 is continuous to the first flow channel. In addition, the figure illustrates a configuration in which the second connector 20a illustrated in FIG. 2 is provided such that the upstream-side divergence flow channel 42c of the integrated flow-channel member 42 is continuous to the second flow channel 21. An outer circumferential surface of a region of the second housing 25a of the second connector 20a which contains the second flow channel 21 (in FIG. 11, the upstream-side divergence flow channel 42c) extends forward (left side on the paper surface) more than the front end portion of the two-divided second housing 25b. The second connector 20a has a projecting structure 52 formed to have an annular shape on the inner circumferential surface in the vicinity of the front end portion of the extended second housing 25a. In addition, the first connector 10a (connector containing the inflow channel 41b of the cell culture vessel 41) is provided with an annular recessed structure 53 at a position at which the recessed structure does not interfere with the first sealing member 17a in the outer circumferential surface of the first housing 14a. During the connection between the cell culture vessel 41 and the integrated flow-channel member 42, the recessed structure 53 engages with the projecting structure 52, and thereby a snap-fit structure is formed. Thus it is possible to prevent the cell culture vessel 41 from being unintentionally detached. Although not illustrated, the outflow channel 41c of the cell culture vessel 41 and the downstream-side divergence flow channel 42d of the integrated flow-channel member 42 have the same configuration as described above.

FIG. 11 illustrates a case where the first connector 10a and the second connector 20b of Example 2 illustrated in FIG. 2 are used as the first connector and the second connector that configure the sterile connector; however, the configuration is not limited thereto. For example, the sterile connector of Example 1 illustrated in FIG. 1, the sterile connector of Example 3 illustrated in FIG. 4, or the sterile connector of Example 4 illustrated in FIG. 5 may be used.

FIG. 12 illustrates views of a modification example of the cell culture vessel and the integrated flow-channel member that configure the cell culture device illustrated in FIG. 7, FIG. 12(a) is a top view of the cell culture vessel, and FIG. 12(b) is a perspective view of the external appearances of the cell culture vessel and an integrated flow-channel member. In FIGS. 7 and 8 described above, in the configuration, the inflow channel 41b and the outflow channel 41c in the cell culture vessel 41 are vertically disposed with the culture surface 41a as a reference, and the upstream-side divergence flow channel 42c and the downstream-side divergence flow channel 42d of the integrated flow-channel member 42 are vertically disposed in the same side surface. The cell culture vessel 41 illustrated in FIG. 12(a) includes the inflow channel 41b and the outflow channel 41c which communicate with the culture surface 41a at two positions on a diagonal line on the culture surface 41a, respectively, on the bottom side of the cylindrical culture surface 41a and extend in parallel in the same horizontal plane in a tangential direction to the culture surface 41a having a circular cross section. In this manner, the inflow channel 41b and the outflow channel 41c are connected to the connection port 41d on the right side and left side in the horizontal direction on the same side surface of the cell culture vessel 41.

In addition, as illustrated in FIG. 12(b), the integrated flow-channel member 42 is provided with slits 55 at positions facing the inflow channel 41b and the outflow channel 41c of the cell culture vessel 41, and positions at which insertion can be performed, respectively, with the entire side surfaces (only one side surface illustrated in FIG. 12(b)) thereof covered with the sealing member 54 formed of the elastic material such as rubber. In FIG. 12(b), although not illustrated, at the position of each slit 55, that is, the slit 55 facing the inflow channel 41b, the connector, which is continuous to the upstream-side divergence flow channel 42b, is disposed inside the integrated flow-channel member 42. Similarly, at the position of the slit 55 facing the outflow channel 41c, the connector, which is continuous to the downstream-side divergence flow channel 42d, is disposed inside the integrated flow-channel member 42. Such a configuration enables the number of components to be reduced, compared to the structure illustrated in FIG. 9. In other words, two sealing members are disposed on the side surfaces of the integrated flow-channel member 42 in FIG. 9; however, in the configuration illustrated in FIG. 12(c), it is possible to reduce the sealing members to one member that is disposed on one side surface. The slits 55 may be formed, corresponding to the number of flow channels which are connected to each other, and three lines of slits 55 or four lines of slits 55 may be formed on the same side surface of the integrated flow-channel member 42.

In this Example, the flow-channel switching member 43 is configured to be provided with the connection flow channel through which the downstream-side common flow channel 42f can be connected to the downstream-side divergence flow channel 42d; however, the configuration is not limited thereto. For example, a configuration of including a connection flow channel that can connect the upstream-side common flow channel 42b and the upstream-side divergence flow channel 42c, or a configuration in which the integrated flow-channel member 42 is disposed on an upper side may be employed.

According to this Example, it is possible to easily detach the desired cell culture vessel from the closed cell culture device while particles containing microorganisms, types of bacteria, or the like are prevented from invading the channel from the outside world.

In addition, according to this Example, the desired cell culture vessel is easily attachable to and detachable from the integrated flow-channel member through only the movement of the vessel in one direction, while the closed system is maintained.

In addition, according to the invention, while the closed system is maintained in the flow channel in the integrated flow-channel member and the flow channel in the cell culture vessel which communicate with each other, it is possible to maintain uniform flow-channel resistance.

The invention is not limited to Examples described above, and includes various modification examples. For example, Examples above are described in detail for easy understanding of the invention, and the invention is not necessarily limited to including the entire configuration described above. In addition, it is possible to replace a configuration of any Example with a part of a configuration of another Example, and it is possible to add a configuration of any Example to a configuration of another Example. In addition, it is possible to perform addition removal replacement of a configuration of any Example to from with a part of a configuration of each of Examples.

### REFERENCE SIGNS LIST

1: cell culture device
10, 10a, 10b: first connector
11: first flow channel
12: first opening
13, 13a, 13b: first pipeline
14, 14a, 14b: first housing
14c: variable protrusion
15: second opening
16: first recessed portion
17, 17a: first sealing member
17A: first slit
18: first connector end
20, 20a: second connector
21: second flow channel
22: third opening
23, 23a, 23b: second sealing member
23A: second slit
24: second recessed portion
25, 25a, 25b: second housing
26: second connector end
31, 41: cell culture vessel
32: supply bag
33: collecting bag
34, 42b: upstream-side common flow channel
35, 42c: upstream-side divergence flow channel
36, 42d: downstream-side divergence flow channel
37, 42f: downstream-side common flow channel
38: flow-channel switching mechanism
39: squeeze pump
41a: culture surface
41b: inflow channel
41c: outflow channel
41d: connection port
41e: entrance
41f: exit
42: integrated flow-channel member
42a: inlet
42e: storage chamber
42g: outlet
42h: port
50: magnet
51: electromagnet
52: projecting structure
53: recessed structure
54: sealing member
55: slit

1. A sterile connector comprising:
a first connector that is provided with a first housing having a first flow channel for causing a fluid to flow therethrough, a first pipeline which is continuous to the first flow channel, a first opening to which one end of the first pipeline is opened, a second opening which is demarcated with an end portion of the first housing, and a first sealing member which covers the second opening with the first opening positioned inward from the second opening in an axial direction of the first housing; and
a second connector that is provided with a second housing having a second flow channel for causing a fluid to flow therethrough, a third opening which is demarcated with an end portion of the second housing, and a second sealing member which covers the third opening,
wherein the first and second connectors are attachable to and detachable from each other,
wherein the first sealing member seals a gap between an outer circumferential surface of the second housing and an inner circumferential surface of the first housing with which the second opening is demarcated, and
wherein the second sealing member seals a gap between an outer circumferential surface of the first pipeline and an inner circumferential surface of the second housing with which the third opening is demarcated, and the first flow channel communicates with the second flow channel through the second sealing member.

2. The sterile connector according to claim 1,
wherein the first sealing member is provided with a first slit at the substantially central portion thereof,
wherein the second sealing member is provided with a second slit at the substantially central portion thereof, and
wherein the second housing is inserted into the first housing via the first slit, and the first pipeline is insertable into the second housing via the second slit.

3. The sterile connector according to claim 1,
wherein the first pipeline has a needle shape, the first pipeline penetrates through the second sealing member, and thereby the first flow channel communicates with the second flow channel.

4. The sterile connector according to claim 2,
wherein the first sealing member has an H-shaped longitudinal section and is provided with disk-shaped portions having two different diameters and a connection portion that connects the two disk-shaped portions, and the first slit is formed to penetrate through the two disk-shaped portions and the connection portion.

5. The sterile connector according to claim 4,
wherein the first housing is divided into two parts outward in the axial direction of the first housing from the first opening, and
wherein an outer edge portion of the disk-shaped portion, which faces the first opening of the first sealing member, is interposed and fixed between the divided first housings.

6. The sterile connector according to claim 5,
wherein the second sealing member has an H-shaped longitudinal section and is provided with two disk-shaped portions having different diameters and a connection portion that connects the two disk-shaped portions, and the second slit is formed to penetrate through the two disk-shaped portions and the connection portion.

7. The sterile connector according to claim 6,
wherein the second housing is divided into two parts on a side of the end portion with which the third opening is demarcated, and
wherein an outer edge of the disk-shaped portion, which faces the second flow channel of the second sealing member, is interposed between the divided second housings, and an inner circumferential surface of one divided second housing, which is disposed on the second flow channel side, has a shape with an inner diameter that is reduced toward the second flow channel.

8. The sterile connector according to claim 2,
wherein the first housing is provided with a variable protrusion that has a variable inner volume with one end communicating with a space formed between the inner circumferential surface of the first housing and the outer circumferential surface of the first pipeline and the other end closed, such that the space enters a positive pressure state or a negative pressure state.

9. The sterile connector according to claim 2,
wherein, in a case where one connector is caused to move in a direction in which the connector is separated from the other connector from a state in which the first and second connectors are connected, the first pipeline is separated from the second sealing member and thereby the second sealing member seals the third opening, and the second housing is separated from the first sealing member and thereby the first sealing member seals the second opening.

10. The sterile connector according to claim 2,
wherein, in a case where one connector is caused to move in a direction in which the connector is separated from the other connector from a state in which the first and second connectors are connected, a pressure state of a fluid in the first flow channel becomes a negative pressure state.

11. A cell culture device comprising:
a cell culture vessel that is provided with an inflow channel through which a liquid for culture circulates and an outflow channel through which the liquid is discharged after use; and
an integrated flow-channel member that is configured to connect a plurality of cell culture vessels in parallel, that is provided with an upstream-side divergence flow channel and a downstream-side divergence flow channel which correspond to the cell culture vessels, and that sends a liquid for culture to any desired cell culture vessel of the plurality of cell culture vessels, that is, to the inflow channel via the upstream-side divergence flow channel,
wherein the integrated flow-channel member is provided with a first housing having a first pipeline that is continuous to each of the upstream-side divergence flow channel and the downstream-side divergence flow channel, a first opening to which one end of the first pipeline is opened, a second opening which is demarcated with an end portion of the first housing, and a first sealing member which covers the second opening,
wherein the cell culture vessel is provided with a second housing having each of the inflow channel and the outflow channel, a third opening which is demarcated with an end portion of the second housing, and a second sealing member which covers the third opening, and
wherein the first sealing member seals a gap between an inner circumferential surface of the first housing and an outer circumferential surface of the second housing, the second sealing member seals a gap between the second housing and an outer circumferential surface of the first pipeline, and the cell culture vessel is connected to the integrated flow-channel member.

12. A cell culture device comprising:
a cell culture vessel that is provided with an inflow channel through which a liquid for culture circulates and an outflow channel through which the liquid is discharged after use; and
an integrated flow-channel member that is configured to connect a plurality of cell culture vessels in parallel, that is provided with an upstream-side divergence flow channel and a downstream-side divergence flow channel which correspond to the cell culture vessels, and that sends a liquid for culture to any desired cell culture vessel of the plurality of cell culture vessels, that is, to the inflow channel via the upstream-side divergence flow channel,
wherein the cell culture vessel is provided with a first housing having a first pipeline that is continuous to each of the inflow channel and the outflow channel, a first opening to which one end of each of the first pipelines is opened, a second opening which is demarcated with an end portion of the first housing, and a first sealing member which covers the second opening,
wherein the integrated flow-channel member is provided with a second housing having each of the upstream-side divergence flow channel and the downstream-side divergence flow channel, a third opening which is demarcated with an end portion of the second housing, and a second sealing member which covers the third opening, and
wherein the first sealing member seals a gap between an inner circumferential surface of the first housing and an outer circumferential surface of the second housing, the second sealing member seals a gap between the second housing and an outer circumferential surface of the first pipeline, and the cell culture vessel is connected to the integrated flow-channel member.

13. The cell culture device according to claim 11,
wherein the first sealing member is provided with a first slit at the substantially central portion thereof,
wherein the second sealing member is provided with a second slit at the substantially central portion thereof, and
wherein the second housing is inserted into the first housing via the first slit, the first pipeline is insertable into the second housing via the second slit, thereby, the inflow channel communicates with the upstream-side divergence flow channel, and the outflow channel communicates with the downstream-side divergence flow channel.

14. The cell culture device according to claim 13,
wherein end portions of the inflow channel and the outflow channel are disposed on one side surface of the cell culture vessel, and end portions of the upstream-side divergence flow channel and the downstream-side divergence flow channel are disposed on the same side surface of the integrated flow-channel member.

15. The cell culture device according to claim 11,
wherein, in a case where a cell culture vessel connected to the integrated flow-channel member is detached, the first pipeline is separated from the second sealing member and thereby the second sealing member seals the inflow channel and the outflow channel of the cell culture vessel, and the second housing is separated from the first sealing member and thereby the first sealing member seals the upstream-side divergence flow channel and the downstream-side divergence flow channel of the integrated flow-channel member.

16. The cell culture device according to claim 12,
wherein, in a case where a cell culture vessel connected to the integrated flow-channel member is detached, the first pipeline is separated from the second sealing member and thereby the second sealing member seals the upstream-side divergence flow channel and the downstream-side divergence flow channel of the integrated flow-channel member, and the second housing is separated from the first sealing member and thereby the first sealing member seals the inflow channel and the outflow channel of the cell culture vessel.

17. The sterile connector according to claim 3,
wherein, in a case where one connector is caused to move in a direction in which the connector is separated from the other connector from a state in which the first and second connectors are connected, a pressure state of a fluid in the first flow channel becomes a negative pressure state.

18. The cell culture device according to claim 12,
wherein the first sealing member is provided with a first slit at the substantially central portion thereof,
wherein the second sealing member is provided with a second slit at the substantially central portion thereof, and
wherein the second housing is inserted into the first housing via the first slit, the first pipeline is insertable into the second housing via the second slit, thereby, the inflow channel communicates with the upstream-side divergence flow channel, and the outflow channel communicates with the downstream-side divergence flow channel.

19. The cell culture device according to claim 18,
wherein end portions of the inflow channel and the outflow channel are disposed on one side surface of the cell culture vessel, and end portions of the upstream-side divergence flow channel and the downstream-side divergence flow channel are disposed on the same side surface of the integrated flow-channel member.
